(19) United States
(12) Patent Application Publication
(10) Pub. No.: US 2024/0084374 A1
(43) Pub. Date: Mar. 14, 2024

(54) METHOD FOR ESTIMATION OF FETAL FRACTION IN CELL-FREE DNA FROM MATERNAL SAMPLE

(71) Applicant: Bio-Rad Laboratories, Inc., Hercules, CA (US)

(72) Inventors: Chenyu LI, Hercules, CA (US); Olga MIKHAYLICHENKO, Hercules, CA (US); Nathan HENDEL, Hercules, CA (US); Anthony HENRIQUEZ, Hercules, CA (US); Richard DANNEBAUM, Hercules, CA (US); Monica HERRERA, Dublin, CA (US); Eric HALL, Hercules, CA (US); Séverine MARGERIDON, Hercules, CA (US); Thea RIEL, Hercules, CA (US)

(21) Appl. No.: 18/465,321

(22) Filed: Sep. 12, 2023

Related U.S. Application Data
(60) Provisional application No. 63/406,194, filed on Sep. 13, 2022.
(60) Provisional application No. 63/472,183, filed on Jun. 9, 2023.

(51) Int. Cl. C12Q 1/686

(52) U.S. Cl. CPC C12Q 1/686; C12Q 2600/154

(57) ABSTRACT

The digital amplification methods and kits provide the ability to estimate the fetal fraction of cell-free DNA (cfDNA) in a maternal sample, e.g., plasma or serum, by analysis of target sites that are differentially methylated in fetal and maternal cfDNA.

### CROSS-REFERENCE TO RELATED PATENT APPLICATIONS

The present patent application claims benefit to each of U.S. Provisional Patent Application No. 63/406,194, filed Sep. 13, 2022 and U.S. Provisional Patent Application No. 63/472,183, filed Jun. 9, 2023, each of which are incorporated by reference for all purposes.

### BACKGROUND

Accurate diagnosis of chromosomal aneuploidies, such as trisomies of chromosomes 13, 18, and 21, requires an accurate estimate of the fetal fraction of cell-free DNA (cfDNA) in maternal blood plasma. Cell-free DNA is present at low levels in maternal whole blood, 0-100 ng/mL, and while the median fetal fraction of cfDNA is around 11% in the first trimester of pregnancy, it can be lower than 4% in the early stages of pregnancy when noninvasive prenatal testing (NIPT) is recommended (Wang et al, Prenat. Diagn. 33:662-666, 2013). Additionally, fetal cfDNA must be distinguished from maternal cfDNA, despite the high similarity in genetic sequence.

### BRIEF SUMMARY

In one aspect, the disclosure provides a digital PCR (dPCR) method for quantifying the fetal fraction cfDNA in maternal blood plasma, utilizing methylation-sensitive restriction enzyme (MSRE) digestion. In some embodiments, the disclosure provides a method of estimating the fraction of fetal DNA in a cfDNA sample obtained from a blood sample from a pregnant human subject. In some embodiments, the method comprises a digital amplification reaction method comprising: (a) partitioning (e.g., distributing) into partitions an amplification reaction mixture comprising cfDNA from the cfDNA sample, amplification reagents, and a plurality of amplification sets comprising primer and probe sets, wherein each amplification set comprises primers and probes for multiplex amplification and each amplification set generates amplification products, when target is present, comprising a distinct label distinguishable from the label for each of the other amplification sets; and wherein the plurality comprises: (i) an amplification set that targets sites that are hypermethylated in fetal DNA and hypomethylated in maternal DNA; and (ii) an amplification set that targets sites that are hypermethylated in maternal DNA and hypomethylated in fetal DNA and optionally one or more of (iii), (iv), and (v); (iii) an amplification set that targets total cfDNA comprising methylation insensitive regions from chromosomes unlikely to exhibit aneuploidy; (iv) an amplification set that targets sites that are hypermethylated in fetal DNA and maternal DNA; (v) an amplification set that targets sites that are hypomethylated in fetal DNA and maternal DNA; (b) incubating the cfDNA with a methylation-sensitive restriction enzyme (MSRE) cocktail comprising at least one methylation-sensitive restriction enzyme that cleaves unmethylated (e.g., hypomethylated) DNA; (c) amplifying in the partitions target nucleic acid sequences, if present, to obtain amplification products; (d) detecting in the partitions a signal from each distinct label from the amplification products; and (e) quantifying the signal for each distinct label. In some embodiments, the plurality comprises (iii) the amplification set that targets total cfDNA comprising methylation insensitive regions from chromosomes unlikely to exhibit aneuploidy. In some embodiments, each of the amplification sets of (i) and (ii) comprises primers and probes to target at least three sites. In some embodiments, wherein each of the amplification sets of (i)-(v) comprises primers and probes to target at least three sites or between 6-10 sites. In some embodiments, the method further comprises employing an amplification set that targets methylation-insensitve regions of the Y chromosome. In additional embodiments, the method can further comprise employing an amplification set that targets sites that are hypomethylated in both fetal and maternal cfDNA; and/or an amplification set that targets sites that are hypermethylated in both fetal and maternal cfDNA. In some embodiments, the amplification reaction mixture further comprises a control target completely methylated synthetic DNA sequence and/or a completely unmethylated version of the same synthetic DNA sequence. In some embodiments, the digital amplification reaction method is a digital PCR method, such as a droplet digital PCR method. In some embodiments, the amplification reaction mixture in the partitioning comprises the MSRE cocktail, and after the incubating occurs after the partitioning and before the amplifying. In some embodiments, step (b) is performed before the partitioning and cfDNA subjected to digestion is added to the amplification reaction mixture. In some embodiments, the MSRE cocktail comprises at least two, at least three, or at least four methylation-sensitive restriction enzymes; and/or wherein the MSRE cocktail comprises a restriction enzyme selected from HhaI, HpaII, AciI, HpyCH4IV, and BsaHI. In some embodiments, the MSRE cocktail comprises at least two, or three of the restriction enzymes HhaI, HpaII, AciI, HpyCH4IV, and BsaHI. In some embodiments, the MSRE cocktail comprises at least HhaI and HpyCH4IV. In some embodiments, the cfDNA sample is obtained from plasma or serum. In some embodiments, each label is a fluorescent label. In some embodiments, the probe is a molecular beacon probe comprising a fluorescent label. In some embodiments, each probe is an oligonucleotide that hybridizes to complementary oligonucleotide that comprises label that provides a detectable signal. In some embodiments, the incubating of the cfDNA with the MSRE cocktail occurs in the partitions. In some embodiments, the incubating of the cfDNA with the MSRE cocktail occurs in a bulk solution before the distributing (a). In some embodiments, the method further comprises determining the normalized copy concentration for each of the targets, for example based on the number of targets in an amplification set (Nᵢ). In some embodiments, method further comprises determining a corrected concentration of fetal cfDNA (Fet_{Corr}) in the cfDNA sample and/or a corrected concentration of maternal cfDNA (Mat_{Corr}) in the cfDNA sample, wherein determining the corrected concentration of fetal cfDNA comprises a calculation:

$$[ {Fet}_{Corr} ] = ( \frac{[ Total ]}{[ Hyper ] - [ Hypo ]} ) * ( [ Fet ] - [ Hypo ] ) , \qquad ( Eq . 1 )$$

wherein [Total] is total cfDNA copy concentration based on signal in partitions from the amplification set that targets total cfDNA comprising methylation insensitive regions from chromosomes unlikely to exhibit aneuploidy, [Hyper] is hypermethylated reference copy concentration based on signal in partitions from the amplification set that targets sites that are hypermethylated in fetal DNA and maternal DNA, [Hypo] is hypomethylated reference copy concentration based on signal in partitions from the amplification set that targets sites that are hypomethylated in fetal DNA and maternal DNA and [Fet] is fetal cfDNA copy concentration based on signal in partitions from the amplification set that targets sites that are hypermethylated in fetal DNA and hypomethylated in maternal DNA and determining the corrected concentration of maternal cfDNA comprises a calculation

$$[ {Mat}_{Corr} ] = ( \frac{[ Total ]}{[ Hyper ] - [ Hypo ]} ) * ( [ Mat ] - [ Hypo ] ) , \qquad ( Eq . 2 )$$

wherein [Mat] is maternal cfDNA copy concentration based on signal in partitions from the amplification set that targets sites that are hypermethylated in maternal DNA and hypomethylated in fetal DNA In some embodiments, the method further comprises determining the fetal fraction (FF) in the cfDNA sample, wherein determining the fetal fraction comprises at least one of the following calculations (a)-(d):

$$FF = \frac{[ {Fet}_{Corr} ]}{[ Total ]} \qquad ( a )$$

$$FF = \frac{[ {Fet}_{Corr} ]}{[ {Fet}_{Corr} ] + [ {Mat}_{Corr} ]} , \qquad ( b )$$

$$FF = 1 - \frac{[ {Mat}_{Corr} ]}{[ Total ]} , or \qquad ( c )$$

$$FF = 1 - \frac{[ {Mat}_{Corr} ]}{[ {Fet}_{Corr} ] + [ {Mat}_{Corr} ]} . \qquad ( d )$$

In some embodiments, the method further comprises computing an estimated fetal fraction at least partially based on the fetal fraction in the cfDNA sample and a model. In some embodiments, the model is a generalized additive model (GAM), a linear model, or a second-order polynomial model at least partially based on a set of clinical fetal fraction data and a corresponding set of fetal fraction measurements using next-generation sequencing (NGS).

In some embodiments, the method further comprises determining the fetal fraction of a male fetus in the cfDNA sample, wherein determining the male fetus fetal fraction comprises:

$$FF = 1 - \frac{[ {Mat}_{Corr} ]}{[ {Fet}_{Corr} ] + [ {Mat}_{Corr} ]} or \qquad ( Eq . 10 )$$

$$FF = \frac{[ YChr ]}{[ To tal ]} or$$

$$FF = \frac{[ YChr ]}{[ {Fet}_{Corr} ] + [ {Mat}_{Corr} ]} ,$$

wherein [YChr] is the concentration of Y-chromosome specific sequences based on signal in partitions from the amplification set that targets methylation-insensitive regions of the Y chromosome

In some embodiments of the methods or kits described herein, the sites that are hypermethylated in fetal DNA and hypomethylated in maternal DNA are sites within one or more or all of:

| Chromosome | Start | End |
|---|---|---|
| chr1 | 10965026 | 10965118 |
| chr1 | 36457885 | 36458000 |
| chr11 | 77152247 | 77152348 |
| chr14 | 100081619 | 100081733 |
| chr14 | 105390470 | 105390583 |
| chr15 | 81781663 | 81781753 |
| chr17 | 75997501 | 75997633 |
| chr2 | 196211575 | 196211683. |

In some embodiments of the methods or kits described herein, the sites that are hypermethylated in maternal DNA and hypomethylated in fetal DNA are sites within one or more or all of:

| Chromosome | Start | End |
|---|---|---|
| chr1 | 110390715 | 110390806 |
| chr19 | 8525569 | 8525674 |
| chr2 | 236576106 | 236576215 |
| chr2 | 239532002 | 239532126 |
| chr2 | 72115414 | 72115526 |
| chr4 | 90104102 | 90104228 |
| chr5 | 142280348 | 142280462. |

In some embodiments of the methods or kits described herein, the sites that are hypermethylated in fetal DNA and maternal DNA are sites within one or more or all of:

| Chromosome | Start | End |
|---|---|---|
| chr19 | 12930395 | 12930501 |
| chr2 | 98903445 | 98903560 |
| chr20 | 50670188 | 50670287 |
| chr4 | 186620052 | 186620153 |
| chr4 | 41181277 | 41181358 |
| chr5 | 133097153 | 133097246 |
| chr6 | 3363036 | 3363147. |

In some embodiments of the methods or kits described herein, the sites that are hypomethylated in fetal DNA and maternal DNA are sites within one or more or all of:

| Chromosome | Start | End |
|---|---|---|
| chr10 | 73744741 | 73744849 |
| chr12 | 104050025 | 104050120 |
| chr17 | 48108167 | 48108297 |
| chr17 | 68292301 | 68292434 |
| chr2 | 27369852 | 27369937 |
| chr2 | 28870489 | 28870604. |

In some embodiments of the methods or kits described herein, the methylation insensitive regions from chromosomes unlikely to exhibit aneuploidy are sites within one or more or all of:

| Chromosome | Start | End |
|---|---|---|
| chr3 | 104042356 | 104042470 |
| chr3 | 104263905 | 104264029 |
| chr3 | 133296976 | 133297077 |
| chr3 | 169019731 | 169019836 |
| chr3 | 17807278 | 17807410 |
| chr3 | 22594415 | 22594519 |
| chr3 | 25620061 | 25620168 |
| chr3 | 35697692 | 35697774 |
| chr3 | 82529219 | 82529320. |

In some embodiments of the methods or kits described herein, the methylation-insensitive regions of the Y chromosome are within one or more or all of:

| Chromosome | Start | End |
|---|---|---|
| chrY | 13817626 | 13817726 |
| chrY | 13861919 | 13862016 |
| chrY | 14225706 | 14225797 |
| chrY | 14405980 | 14406112 |
| chrY | 15558950 | 15559068 |
| chrY | 16701419 | 16701541 |
| chrY | 17109321 | 17109424 |
| chrY | 19228514 | 19228627 |
| chrY | 19731092 | 19731204 |
| chrY | 21046467 | 21046568. |

In a further aspect, the disclosure provides a digital amplification kit for estimating the fraction of fetal DNA in a cfDNA sample obtained from a plasma or serum sample from a pregnant human subject, the kit comprising:
(a) an amplification reaction mixture comprising amplification reagents, and a plurality of amplification sets comprising primer and probe sets, wherein each amplification set comprises a distinct label distinguishable from the label for each of the other sets, and each set comprises primers and probes for multiplex amplification, and wherein the plurality of comprises:
(i) an amplification set that targets sites that are hypermethylated in fetal DNA and hypomethylated in maternal DNA; and
(ii) an amplification set that targets sites that are hypermethylated in maternal DNA and hypomethylated in fetal DNA; and optionally one or more of (iii), (iv), and (v);
(iii) an amplification set that targets total cfDNA comprising methylation insensitive regions from chromosomes unlikely to exhibit aneuploidy;
(iv) an amplification set that targets sites that are hypermethylated in fetal DNA and maternal DNA;
(v) an amplification set that targets sites that are hypomethylated in fetal DNA and maternal DNA. In some embodiments, each of the amplification sets of (i) and (ii) comprises primers and probes to target at least three sites. In some embodiments, the kit further comprises an amplification set that targets methylation-insensitve regions of the Y chromosome. In some embodiments, the kit further comprises a completely methylated synthetic sequence of DNA and/or a completely unmethylated version of the same synthetic sequence. In some embodiments, each label is a fluorescent label. In some embodiments, the probe is a molecular beacon probe comprising a fluorescent label. In some embodiments, each probe is an oligonucleotide that hybridizes to complementary oligonucleotide that comprises label that provides a detectable signal. In some embodiments, the kit further comprises a methylation-sensitive restriction enzyme (MSRE) cocktail comprises at least one MSRE that cleaves hypomethylated DNA. In some embodiments, the MSRE cocktail comprises at least two, at least three, or at least four methylation-sensitive restriction enzymes; and/or wherein the MSRE cocktail comprises a restriction enzyme selected from HhaI, HpaII, AciI, HpyCH4IV, and BsaHI. In some embodiments, the kit comprises a methylation-sensitive restriction enzyme (MSRE) cocktail comprising at least two, three, or more of the restriction enzymes HhaI, HpaII, AciI, HpyCH4IV, and BsaHI. In some embodiments, the MSRE cocktail comprises at least HhaI and HpyCH4IV. In some embodiments, the kit further comprises a methylation-sensitive restriction enzyme (MSRE) cocktail comprising at least two, three, or more of the restriction enzymes HhaI, HpaII, AciI, HpyCH4IV, and BsaHI.

In one aspect, a dataset of calculated fetal fractions based on the disclosed methods and corresponding measured fetal fractions using next-generation sequencing (NGS) of different NIPT clinical samples from human subjects is created. Several models are developed that could take into account not only the calculated fetal fractions and corresponding NGS measurements but also parameters such as gestation week and the Y chromosome calculations.

### Terminology

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization described below are those well-known and commonly employed in the art. Standard techniques are used for nucleic acid and peptide synthesis. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document. The nomenclature used herein and the laboratory procedures in analytical chemistry, and organic synthetic described below are those well-known and commonly employed in the art.

The term “cell-free DNA sample” or “cfDNA sample” refers to a nucleic acid sample comprising extracellular DNA, which nucleic acid sample is obtained from any cell-free biological fluid, for example, whole blood processed to remove cells, urine, saliva, or other biological fluid. In typical embodiments, cfDNA for analysis is obtained from whole blood processed to remove cells, e.g., a plasma or serum sample. As used herein, the term “cfDNA” thus refers to DNA recoverable from the non-cellular fraction of a bodily fluid, such as blood.

The methylation status refers to the presence of methyl groups at a particular DNA sequence. In some embodiments, methylation of DNA refers to the presence or absence of methylcytosine at one or more CpG dinucleotides in a DNA sequence. The term “methylation state” or “methylation status” with respect at CpG dinucleotide methylation refers to the presence or absence of 5-methylcytosine (“5-mC” or “5-mCyt”) at one or a plurality of CpG dinucleotides within a DNA sequence. Methylation states at one or more particular methylation sites within a DNA sequence include “unmethylated,” “fully-methylated,” and “hemi-methylated.” For purposes of the present application, the term “hypermethylation” refers to a region where the average frequency of methylation for a particular subset of samples, e.g., fetal DNA or maternal DNA, is greater than 80% as determined by methylation-based sequencing. “Hypomethylation” refers to a region where the average frequency of methylation for a particular subset of samples, e.g., fetal DNA or maternal DNA, is less than 20% as determined by methylation-based sequencing.

A methylation-sensitive restriction enzyme (MSRE) refers to an enzyme that cleaves DNA at specific unmethylated cytosine residues, but does not cleave at the recognition sequence when the cytosine residues are methylated.

As used herein, a “methylation-sensitive” genomic region refers to a genomic DNA that can be methylated, e.g., at CpG sequences, such that the site is not cleavable by a methylation sensitive restriction enzyme in a methylated state and cleavable when methylation is not present. “Cleavable” as used herein means that at least 50% of the DNA is digested with the methylation-sensitive restriction enzyme when the recognition sequence is unmethylated compared to when it is methylated. Accordingly, detection of an amplification product obtained from amplification of cfDNA that comprises a methylation site following digestion with MSRE means that the cfDNA is methylated at that site.

The term “amplification reaction” refers to any in vitro means for multiplying the copies of a target sequence of nucleic acid in a linear or exponential manner. Such methods include but are not limited to two-primer methods such as polymerase chain reaction (PCR); ligase methods such as DNA ligase chain reaction (see U.S. Pat. Nos. 4,683,195 and 4,683,202; PCR Protocols: A Guide to Methods and Applications (Innis et al., eds, 1990)) (LCR); QBeta RNA replicase and RNA transcription-based amplification reactions (e.g., amplification that involves T7, T3, or SP6 primed RNA polymerization), such as the transcription amplification system (TAS), nucleic acid sequence based amplification (NASBA), and self-sustained sequence replication (3 SR); isothermal amplification reactions (e.g., single-primer isothermal amplification (SPIA)); as well as others known to those of skill in the art.

“Amplifying” refers to a step of submitting a solution to conditions sufficient to allow for the amplification of a polynucleotide if all the components of the reaction are intact. Components of an amplification reaction include, e.g., primers, a polynucleotide template, polymerase, nucleotides, and the like. In some embodiments, “amplifying” refers to PCR amplification using a first and a second amplification primer.

A “primer” refers to a polynucleotide sequence that hybridizes to a sequence on a target nucleic acid and serves as a point of initiation of nucleic acid synthesis. Primers can be of a variety of lengths and are often less than 100 nucleotides in length, for example 18-55 nucleotides in length. The length and sequences of primers for use in an amplification reaction such as PCR can be designed based on principles known to those of skill in the art. Primers can be DNA, RNA, or a chimera of DNA and RNA portions. In some cases, primers can include one or more modified or non-natural nucleotide bases. In some cases, primers are labeled. In some instances, a primer may also contain a nucleic acid sequence that is not involved in hybridization to the target for amplification, for example, a sequence that hybridizes to another oligonucleotide that is labeled, or a sequence that hybridizes to a capture oligonucleotide, or a tag sequence such as a barcode.

A nucleic acid, or portion thereof, “hybridizes” to another nucleic acid under conditions such that non-specific hybridization is minimal at a defined temperature in a physiological buffer. In some cases, a nucleic acid, or portion thereof, hybridizes to a conserved sequence shared among a group of target nucleic acids. In some cases, a primer, or portion thereof, can hybridize to a primer binding site if there are at least about 6, 7, 8, 9, 10, 11, 12, 13, 14, 15, 16, 17, 18, 19, 20, 21, 22, 23, 24, 25, 26, 27, 28, 29, or 30 contiguous complementary nucleotides, including “universal” nucleotides that are complementary to more than one nucleotide partner. Alternatively, a primer, or portion thereof, can hybridize to a primer binding site if there are fewer than 1 or 2 complementarity mismatches over at least about 12, 13, 14, 15, 16, 17, 18, 19, 20, 25, or 30 contiguous complementary nucleotides. In some embodiments, the defined temperature at which specific hybridization occurs is room temperature. In some embodiments, the defined temperature at which specific hybridization occurs is higher than room temperature. In some embodiments, the defined temperature at which specific hybridization occurs is at least about 37, 40, 42, 45, 50, 55, 60, 65, 70, 75, or 80° C., e.g., about 45° C. to about 60° C., e.g., about 55° C.-59° C. In some embodiments, the defined temperature at which specific hybridization occurs is about 5° C. below the calculated melting temperature of the primers.

As used herein, “nucleic acid” means DNA, RNA, single-stranded, double-stranded, or more highly aggregated hybridization motifs, and any chemical modifications thereof. Modifications include, but are not limited to, those providing chemical groups that incorporate additional charge, polarizability, hydrogen bonding, electrostatic interaction, points of attachment and functionality to the nucleic acid ligand bases or to the nucleic acid ligand as a whole. Such modifications include, but are not limited to, peptide nucleic acids (PNAs), phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases, isocytidine and isoguanidine and the like. Nucleic acids can also include non-natural bases, such as, for example, nitroindole. Modifications can also include 3′ and 5′ modifications including but not limited to capping with a fluorophore (e.g., quantum dot) or another moiety.

A “polymerase” refers to an enzyme that performs template-directed synthesis of polynucleotides, e.g., DNA. The term encompasses both the full length polypeptide and a domain that has polymerase activity. At least five families of DNA-dependent DNA polymerases are known, although most fall into families A, B and C. DNA polymerases are well-known to those skilled in the art. DNA polymerases for use in the compositions and methods disclosed herein can be any polymerase capable of replicating a DNA molecule. In some embodiments, the DNA polymerase is a thermostable polymerase. Thermostable polymerases are isolated from a wide variety of thermophilic bacteria, such as Thermus aquaticus (Taq), Pyrococcus furiosus (Pfu), Pyrococcus woesei (Pwo), Bacillus sterothermophilus (Bst), Sulfolobus acidocaldarius (Sac) Sulfolobus solfataricus (Sso), Pyrodictium occultum (Poc), Pyrodictium abyssi (Pab), and Methanobacterium thermoautotrophicum (Mth), as well as other species. DNA polymerases are known in the art and are commercially available. In some embodiments, the DNA polymerase is Taq, Tbr, Tfl, Tru, Tth, Tli, Tac, Tne, Tma, Tih, Tfi, Pfu, Pwo, Kod, Bst, Sac, Sso, Poc, Pab, Mth, Pho, ES4, VENT™, DEEPVENT™, or an active mutant, variant, or derivative thereof. In some embodiments, the DNA polymerase is Taq DNA polymerase. In some embodiments, the DNA polymerase is a high fidelity DNA polymerase (e.g., iProof™ High-Fidelity DNA Polymerase, Phusion® High-Fidelity DNA polymerase, Q5® High-Fidelity DNA polymerase, Platinum® Taq High Fidelity DNA polymerase, Accura® High-Fidelity Polymerase). In some embodiments, the DNA polymerase is a fast-start or hot-start polymerase (e.g., FastStart™ Taq DNA polymerase, FastStart™ High Fidelity DNA polymerase, or iTaq™ DNA polymerase).

As used herein, the term “partitioning” or “partitioned” refers to separating a sample into a plurality of portions, or “partitions”. Partitions are generally physical, such that a sample in one partition does not, or does not substantially, mix with a sample in an adjacent partition. Partitions can be solid or fluid. In some embodiments, a partition is a solid partition, e.g., a microchannel or microwell. In some embodiments, a partition is a fluid partition, e.g., a droplet. In some embodiments, a fluid partition (e.g., a droplet) is a mixture of immiscible fluids (e.g., water and oil). In some embodiments, a fluid partition (e.g., a droplet) is an aqueous droplet that is surrounded by an immiscible carrier fluid (e.g., oil). Exemplary array of wells and well descriptions can be found for example in U.S. Pat. Nos. 9,103,754 and 10,391,493. The array of wells (set of nanowells, microwells, wells) can function to capture the solid supports, optionally in addressable, known locations. As such, the array of wells can be configured to facilitate bead capture in at least one of a single-solid support format or optionally in small groups of solid supports. Exemplary microwell arrays and methods of delivery of beads to the microwells and analysis thereof is described in, e.g., PCT/US2021/034152.

An “oligonucleotide” is a polynucleotide. In many embodiments, oligonucleotides will have fewer than 250 nucleotides, in some embodiments, between 4-200, e.g., 10-150 nucleotides.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a bead” includes a plurality of such beads and reference to “the sequence” includes reference to one or more sequences known to those skilled in the art, and so forth.

### BRIEF DESCRIPTION OF THE DRAWINGS

FIG. 1 shows methylation-sensitive digestion ddPCR & Y chromosome ddPCR fetal fraction estimations. This FIG. shows the correlation between the methylation-sensitive fetal fraction estimations and the SRY-based fetal fraction estimations for an illustrative assay detailed herein including the Examples section.

FIGS. 2A-2D illustrate an analysis of methylation patterns for maternal and fetal methylation sites as well as ubiquitously methylated or unmethylated sites.

FIG. 3 depicts how methylation-sensitive restriction enzymes (MSREs) enable ddPCR to quantify fetal and maternal cfDNA based on differences in fetal and maternal cfDNA methylation. As depicted, MSRE digestion is performed in-droplet, with no disruption to the ddPCR workflow. Fetal and maternal cfDNA are quantified simultaneously in the same ddPCR reaction. 1) Hypermethylated fetal cfDNA is quantified after MSRE digestion of hypomethylated maternal cfDNA. 2) Maternal cfDNA is quantified after MSRE digestion of hypomethylated fetal cfDNA. 3) Total cfDNA is quantified from non-digested regions.

FIGS. 4A-B depict multiplexed ddPCR assays. FIG. 4A depicts a general ddPCR assay format. FIG. 4B depicts that multiplex primer pairs targeting the same chromosome are combined in a single fluorescent channel using a unique universal probe.

FIGS. 5A-D depict fetal fraction estimation with the developed linear model, polynomial model, and generalized additive model (GAM) described herein including in Example 2. FIG. 5A depicts the fetal fraction estimation with the developed linear model (“LM”) and using prior methods (“FF_calculated”), compared to fetal fraction estimation using next-generation sequencing (NGS). FIG. 5B depicts the calculated fetal fraction with respect to fetal/hyper and maternal/hyper variables. The residual plots indicates a non-linear relationship of fetal/hyper (510) compared with maternal/hyper (512). FIG. 5C depicts the fetal fraction estimation with the order-2 polynomial model (“poly2LM”), compared to fetal fraction estimation using NGS. FIG. 5D depicts the GAM for fetal fraction estimation, compared to fetal fraction estimation using NGS. MPAE, mean average percentage error. MSE, mean squared error.

### DETAILED DESCRIPTION OF THE INVENTION

### Introduction

The present disclosure provides a method of estimating the fraction of fetal DNA in a cfDNA sample obtained from a maternal cell-free biological sample. As described herein, the method comprising a digital amplification reaction comprising evaluating cfDNA obtained from a pregnant subject, typically a human subject, to determine the methylation status, using methylation-sensitive restriction enzymes, of loci that are differentially methylated in fetal vs maternal cfDNA. In particular, the method comprises:
analyzing the methylation status of one or more sites in fetal and maternal cfDNA that are hypermethylated in fetal DNA and hypomethylated in maternal DNA; and detecting the methylation status of one or more sites that are hypermethylated in maternal DNA and hypomethylated in fetal DNA. In some embodiments, the method further comprises amplifying sites that target total cfDNA comprising methylation insensitive genomic regions from chromosome unlikely to exhibit aneuploidy, which provide the ability to quantify the total concentration of DNA, i.e., both fetal and maternal, in the sample. In some embodiments, the method further comprises detection of methylation-insensitive regions of the Y chromosome, if desired, e.g., to determine the fetal sex. In some embodiments, the method further comprises evaluating sites that are hypomethylated in both fetal and maternal cfDNA and/or evaluating sites that are hypermethylated in both fetal and maternal cfDNA for use as internal controls for MSRE digestion. Assessing the level of these various chromosome regions, which have differing methylation profiles, thus provides the ability to quantify the fraction of cfDNA from the maternal sample that arises from the fetus. The estimation of the fetal fraction is important to quality check and predict fetal aneuploidy in a non-invasive prenatal test.

In some embodiments, a fetal fraction calculated/determined according to the methods of the present disclosure may be used in methods for assessing fetal aneuploidy (e.g., trisomies, such as of chromosomes 13, 18 or 21), chromosomal deletions (e.g., microdeletions such as in chromosome 22), or other genomic alterations (e.g., gene mutations associated with diseases such as alpha- or beta-thalassemia, cystic fibrosis, sickle cell anemia, or hemophilia A). For example, the fetal fraction calculated/determined according to the methods of the present disclosure may be used for quality control in such methods.

### Components

In the present disclosure, cell-free DNA obtained from a pregnant subject is evaluated to determine the quantity of fetal cfDNA in maternal blood, i.e., the fraction of cfDNA in maternal blood that is from the fetus. A cfDNA sample from the pregnant subject is digested with one or more methylation-sensitive restriction enzymes followed by amplification of a plurality of target loci that have differing methylation profiles in fetal vs maternal DNA. The fraction of fetal cfDNA can be calculated based on the levels of differentially methylated DNA.

### CfDNA

Cell-free DNA for use in the invention is obtained from a biological fluid sample, typically a blood sample, that is free of cells. Thus, in typical embodiments, the sample is a plasma or serum sample. Isolation of cfDNA can be achieved using any number of different methodologies, e.g., by employing columns or magnetic beads, or other isolation procedures. Kits for extracting cfDNA from samples are commercially available, e.g., from Qiagen, Beckman (e.g., Apostle™ kit), and ThermoFisher (e.g., MagMax™ kit), among others.

### Methylation-Sensitive Restriction Enzymes

The cfDNA is subject to digesting with one or more methylation sensitive restriction enzymes (MSRE). Such enzymes will digest unmethylated regions of DNA, but not methylated regions. Accordingly, amplification products from hypermethylated regions of DNA will be greater in abundance than those from hypomethylated regions of DNA.

In some embodiments, one restriction enzyme is employed. In alternative embodiments, two MSREs are employed. In other embodiments, at least three MSRE are employed. In other embodiments, at least four MSREs are employed. Illustrative restriction enzymes include AatII, AciI, AclI, AfeI, AgeI, AscI, BmgBI, BsaAI, BsaHI, BspDI, ClaI, EagI, FseI, PauI, HhaI, HpaII, HpyCH4IV, HinPII, MluI, NarI, NotI, NruI, PvuI, SacII, and SalI, and SmaI. In some embodiments, one or more of HhaI, HpaII, AciI, and HpyCH4IV is employed in the analysis.

In some embodiments, digestion with the one or more MSREs is performed in bulk prior to distribution of the reaction mixture to partitions as detailed below. However, in preferred embodiments, the cfDNA is added to the dPCR reaction mix along with the PCR reagents for the target amplification sites and the one or more restriction enzymes. Restriction enzyme digestion can then be performed in the partitions, but before amplification.

Further, targets within the cfDNA eluate may be pre-amplified following bulk MSRE digestion, for example, to reduce the amount of assay multiplexing needed to attain sufficient sensitivity and precision.

### Amplification Targets

Determination of fetal fraction typically comprises multiplex amplification of each of the targeted hypomethylated or hypermethylated site in the genomes that are evaluated. Thus, in some embodiments, at least two sites, or at least three sites, or at least four sites or more are targeted for each of the categories of DNA that may be employed in an assay, i.e., sites that are hypermethylated in fetal cfDNA, sites that are hypomethylated in fetal cfDNA, sites that are hypermethylated in maternal cfDNA, sites that are hypomethylated in maternal cfDNA, sites from methylation-insensitive regions of chromosomes unlikely to exhibit aneuploidy, sites from methylation-insensitive regions of the Y chromosome, sites that are hypomethylated in both fetal and maternal cfDNA and sites that are hypermethylated in both fetal and maternal cfDNA.

Differentially methylated sites in maternal compared to fetal cfDNA have been described (see, e.g., Ionnides, Mol. Genet. Genomic Med. 8:e1094, 2020; Hatte et al., PLOS ONE DOI:10.1371/journal.pone.012891, 2015; Bunce et al, Prenat. Diagn. 32:542-54, 2012; Xiang et al, Mol Hum Reprod 20:875-884, 2014). See also, Hatt et al, PLOS ONE, Jul. 31, 2015, pages 1-12; DoI:10.1371/journal.pone.0128918. Differentially methylated sites can also be determined. For example, methylation-based sequencing can be used to identify hypermethylated vs. hypomethylated sequences from a collection of fetal, maternal, and non-pregnant samples. Exemplary target sites are listed in Table 6 and one can use one, some of all of the target sites listed in Table 6, optionally with other target sites not listed in Table 6.

Sites from chromosomes that are unlikely to exhibit aneuploidy can be from an autosome other than chromosome 21, chromosome 13, or chromosome 18. In some embodiments, the sites are from chromosome 3. Methylation-insensitive regions refer to regions of the chromosome not containing CpG sites and thus are unlikely to be methylated in any cell and lack a recognition sequence for the MSRE employed in the method, meaning these sites will not be cleaved by the MSRE even though they are unmethylated.

Sites from methylation-insensitive regions of the Y chromosome refers to methylation-insensitive sequences that are unique to the Y-chromosome such that their detection indicates the presence of a Y-chromosome, i.e., a male fetus.

For purposes of this application, a differentially methylated site is one where the methylation pattern between fetal and maternal DNA is statistically different by two-sample Kolmogorov-Smirnov test (p<0.015). Furthermore, for the selection of methylated sites in fetal cfDNA, sites are selected that have a greater than 80% average methylation frequency in fetal cfDNA, i.e., are hypermethylated; and have less than 20% average methylation frequency, i.e., are hypomethylated, in maternal DNA. Similarly, for the selection of methylated sites in maternal cfDNA, sites are selected that have a greater than 80% average methylation frequency in maternal cfDNA, i.e., are hypermethylated; and have less than 20% average methylation frequency, i.e., are hypomethylated, in fetal DNA.

For selection of hypermethylated target sites as reference sites, sites are selected that have greater than 80% average methylation frequency in both fetal cfDNA and maternal cfDNA.

For selection of hypomethylated target sites as reference sites, sites are selected that have less than 20% average methylation frequency in both fetal cfDNA and maternal cfDNA.

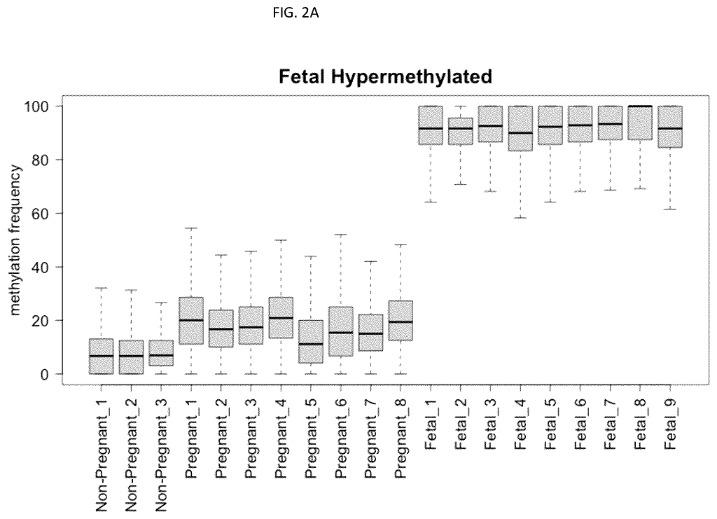

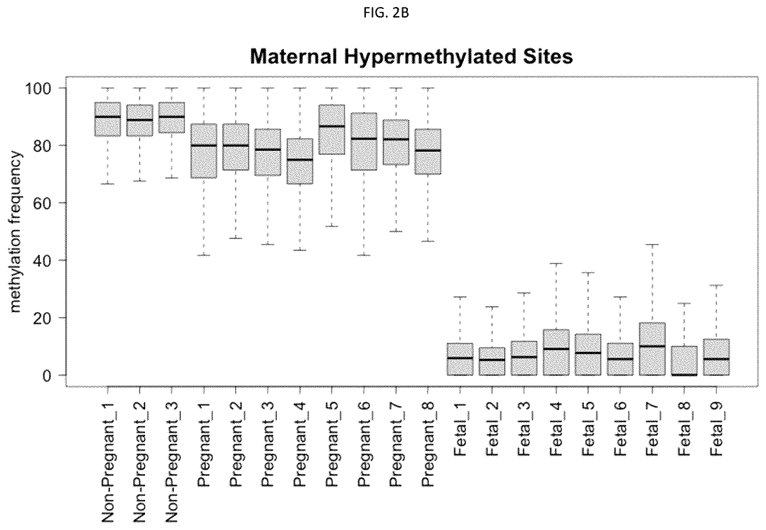

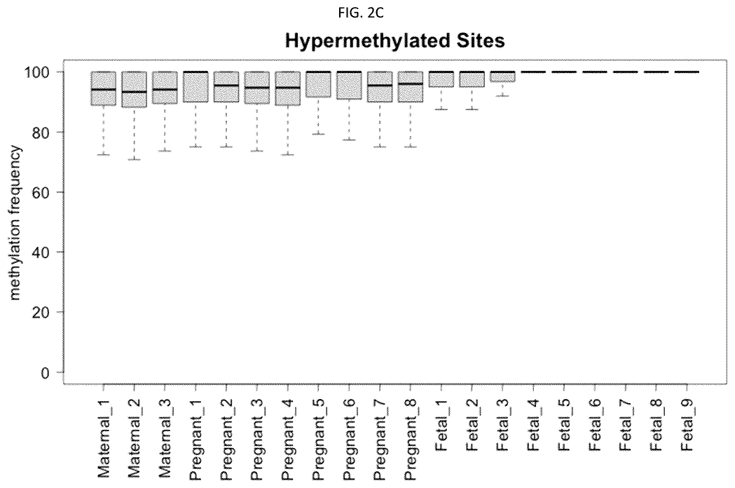

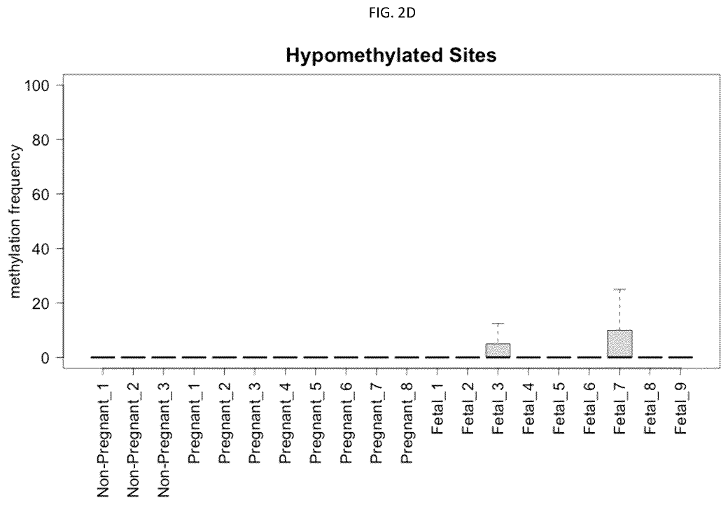

By way of illustration, results of such selections based on analysis of differences in fetal and maternal methylation for selection of fetal hypermethylated, maternal hypermethylated, reference hypermethylated, and reference hypomethylated sites are provided in FIGS. 2A-2D, respectively. In some embodiments, target sites from Table 6 are assayed according to the methods described herein.

### Primers and Probes

Primer and probe sequences for detection of amplified product for a desired target can be designed based on known principles. Amplified products are detected with a detectable label. One of skill understands that there are any number of labeling configurations for the detection of amplified products. In some embodiments, an oligonucleotide is labeled with a detectable agent such as a fluorescent agent, phosphorescent agent, chemiluminescent agent, and the like.

In some embodiments, a probe is labeled, e.g., with a fluorescent label. In alternative embodiments, at least one of a pair of amplification primers is labeled with a detectable label, e.g., a fluorescent label. In some embodiments, a complementary oligonucleotide that hybridizes to a non-target region of a primer or probe is labeled with a detectable label, e.g., a fluorescent label.

In some embodiments, the probe is a TAQMAN™ probe, a SCORPION™ probe, an ECLIPSE™ probe, a molecular beacon probe, a double-stranded probe, a dual hybridization probe, or a double-quenched probe.

In some embodiments, an oligonucleotide, e.g., a primer or probe, is labeled with a detectable label, e.g., a fluorescent label. In some embodiments, the agent is a fluorophore. A large number of fluorophores are available, including from commercial vendors. Non-limiting examples of fluorophores include cyanines (e.g., Cy3, Cy5), indocarbocyanines (e.g., Quasar® 570, Quasar® 670, and Quasar® 705), fluoresceins (e.g., 5′-carboxyfluorescein (FAM), 6-carboxyfluorescein (6-FAM), 5- and 6-carboxyfluorescein (5,6-FAM), 2′-chloro-7′phenyl-1,4-dichloro-6-carboxy-fluorescein (VIC), 6-carboxy-4′-, 5′-dichloro-2′-, 7′-dimethoxy-fluorescein (JOE), 4,7,2′,4′,5′,7′-hexachloro-6-carboxy-fluorescein (HEX), 4,7,2′,7′-tetrachloro-6-carboxy-fluorescein (TET), 2′-chloro-5′-fluoro-7′,8′-benzo-1,4-dichloro-6-carboxyfluorescein (NED), Oregon Green, and Alexa 488), rhodamines (e.g., N,N,N′,N′-tetramethyl-6-carboxyrhodamine (TAMRA) and 5- and 6-carboxy-X-rhodamine (ROX)), tetramethyl rhodamine, and tetramethyl rhodamine isothiocyanate (TRITC)), an Atto dye, eosin, coumarins, pyrenes, tetrapyrroles, arylmethines, and oxazines. In some embodiments, the dye is selected from Cy3, Cy5, Cy4.4, ROX, Atto, FAM, HEX, JOE, QUASAR, rhodamine, TAMRA, TET, Texas Red, TYE, and VIC.

In some embodiments, detection of amplification products is performed via reporter-quencher pairs. Reporter-quencher pairs can be selected from xanthene dyes including fluoresceins and rhodamine dyes. Many suitable forms of these compounds are available commercially with substituents on the phenyl groups, which can be used as the site for bonding or as the bonding functionality for attachment to an oligonucleotide. Another group of fluorescent compounds for use as reporters are the naphthylamines, having an amino group in the alpha or beta position. Included among such naphthylamino compounds are 1-dimethylaminonaphthyl-5 sulfonate, 1-anilino-8-naphthalene sulfonate and 2-p-touidinyl-6-naphthalene sulfonate. Other dyes include 3-phenyl-7-isocyanatocoumarin; acridines such as 9-isothiocyanatoacridine; N-(p-(2-benzoxazolyl)phenyl)maleimide; benzoxadiazoles; stilbenes; pyrenes and the like.

Suitable examples of quenchers can be selected from 6-carboxy-tetramethyl-rhodamine, 4-(4-dimethylaminophenylazo) benzoic acid (DABYL), tetramethylrhodamine (TAMRA), BHQ-O™, BHQ-1 ™, BHQ-2™, and BHQ-3™, each of which are available from Biosearch Technologies, Inc. of Novato, Calif., Qy7™ QSY-9™, QSY-21™ and QSY-35™, each of which are available from Molecular Probes, Inc, and ZEN™ and TAO™ Double-Quenched Probes from Integrated DNA Technologies. Fluorescent and dark quenchers and their relevant optical properties from which exemplary reporter-quencher pairs can be selected are listed and described, for example, in R. W. Sabnis, HANDBOOK OF FLUORESCENT DYES AND PROBES, John Wiley and Sons, New Jersey, 2015.

Primers can be designed taking into consideration the recognition sequences of the one or more MSREs employed for the reaction. Thus, for example, primers and target regions to be amplified are selected to avoid the presence of the one or more MSRE recognition sequences in the primer and/or in amplicons generated during the amplification reactions.

In some embodiments, multiple amplicons can be detected with the same color probe. For example, in some embodiments, 2-20, e.g., 6-10 amplicons are detected with a single-color probe. In some embodiments, multiple color signals are used. For example in some embodiments, one color signal is used to detect regions that are hypermethylated in fetal cfDNA compared to maternal cfDNA, a second color signal is used to detect regions that are hypermethylated in maternal cfDNA compared to fetal cfDNA, and optionally a third color can be used to detect Y-chromosome-specific sequences if present. Additional colors can be used to detect regions that are hypermethylated in both fetal cfDNA and maternal cfDNA, regions that are hypomethylated in both fetal cfDNA and maternal cfDNA, or controls to measure the assay functioned.

### Additional Amplification Reaction Components

The reagent mixture can further comprise additional reagents, e.g., amplification reagents, including for example, one or more of buffers, salts, nucleotides, stabilizers, primers, polymerases, or nuclease-free water. In some embodiments, an additive, e.g., tetramethylammonium chloride (TMAC), DMSO, DTT, or betaine, may be used to enhance amplification specificity or yield.

### Partitioning

Distributing a reaction mixture, e.g., cfDNA, digital amplification reaction components, and the methylation sensitive restriction enzymes, into partitions can be achieved by any methods available. In some embodiments, methods and compositions for delivering reagents to one or more partitions include microfluidic methods using microwell plate, capillaries, oil emulsions, and arrays of miniaturized chambers for partitioning. In some embodiments, partitioning employs droplets. Methods of producing such droplets include droplet or microcapsule merging, coalescing, fusing, bursting, or degrading (e.g., as described in U.S. 2015/0027,892; US 2014/0227,684; WO 2012/149,042; and WO 2014/028,537); droplet injection methods (e.g., as described in WO 2010/151,776); and combinations thereof. Thus, for example, in methods in which the partitions are droplets, one can form droplets as an emulsion with an immiscible fluid such as oil such that the bulk solution forms droplets that contain reaction mixture reagents, including cfDNA template. Methods of emulsion formation are described, for example, in patent applications WO 2011/109546 and WO 2012/061444.

In some embodiments, the amplification reaction is a droplet digital PCR reaction. Methods for performing PCR in droplets are described, for example, in US 2014/0162266, US 2014/0302503, and US 2015/0031034, the contents of each of which is incorporated by reference. In some embodiments, the QX600 Droplet Digital PCR (ddPCR) System (Bio-Rad) is used.

In some embodiments, a detection reagent or a detectable label in the partitions can be detected using any of a variety of detector devices. Exemplary detection methods include optical detection (e.g., fluorescence, or chemiluminescence). As a non-limiting example, a fluorescent label can be detected using a detector device equipped with a module to generate excitation light that can be absorbed by a fluorophore, as well as a module to detect light emitted by the fluorophore.

In some embodiments, the detector further comprises handling capabilities for the partitioned samples (e.g., droplets), with individual partitioned samples entering the detector, undergoing detection, and then exiting the detector. In some embodiments, partitioned samples (e.g., droplets) can be detected serially while the partitioned samples are flowing. In some embodiments, partitioned samples (e.g., droplets) are arrayed on a surface and a detector moves relative to the surface, detecting signal(s) at each position containing a single partition. Examples of detectors are provided in WO 2010/036352, the contents of which are incorporated herein by reference. In some embodiments, detectable labels in partitioned samples can be detected serially without flowing the partitioned samples (e.g., using a chamber slide).

Following acquisition of fluorescence detection data, a general purpose computer system (referred to herein as a “host computer”) can be used to store and process the data. A computer-executable logic can be employed to perform such functions as subtraction of background signal, assignment of target and/or reference sequences, and quantification of the data. A host computer can be useful for displaying, storing, retrieving, or calculating fetal fraction in the sample; storing, retrieving, or calculating raw data from the nucleic acid detection; or displaying, storing, retrieving, or calculating any sample or source information useful in the methods.

The host computer can be configured with many different hardware components and can be made in many dimensions and styles (e.g., desktop PC, laptop, tablet PC, handheld computer, server, workstation, mainframe). Standard components, such as monitors, keyboards, disk drives, CD and/or DVD drives, and the like, can be included. Where the host computer is attached to a network, the connections can be provided via any suitable transport media (e.g., wired, optical, and/or wireless media) and any suitable communication protocol (e.g., TCP/IP); the host computer can include suitable networking hardware (e.g., modem, Ethernet card, WiFi card). The host computer can implement any of a variety of operating systems, including UNIX, Linux, Microsoft Windows, MacOS, or any other operating system.

Computer code for implementing aspects of the present invention can be written in a variety of languages, including PERL, C, C++, Java, JavaScript, VBScript, AWK, or any other scripting or programming language that can be executed on the host computer or that can be compiled to execute on the host computer. Code can also be written or distributed in low level languages such as assembler languages or machine languages.

Scripts or programs incorporating various features of the present invention can be encoded on various computer readable media for storage and/or transmission. Examples of suitable media include magnetic disk or tape, optical storage media such as compact disk (CD) or DVD (digital versatile disk), flash memory, and carrier signals adapted for transmission via wired, optical, and/or wireless networks conforming to a variety of protocols, including the Internet.

### Calculation of Fetal Fraction

Data processing can be used to take detected copy concentrations of fetal-specific or maternal-specific target sites to produce a fetal fraction calculation, optionally as well as a determination of fetal sex (when desired). Table 3 illustrates the various copy concentrations supplied by an exemplary six assay collection across six illustrative fluorescence channels. As noted herein, copy concentrations from one or more fetal-specific or maternal-specific target sites, as well as various control target sites can be determined in partitions from a sample. Thus, for example, fetal cfDNA copy concentration can be determined from one or more target sites that are specifically hypermethylated in fetal cells and hypomethylated in maternal cells, cleaved with one or more MSREs, and detected for example by a probe in a digital assay. Signal for one or more target sites can be multiplexed such that for example probes that detect different fetal hypermethylated target sites have the same color probe, and the sum of partitions having signal of that color divided by the number of targets indicates the fetal copy concentration.

In some embodiments, prior to any downstream calculation, each of the copy concentrations (e.g., each of the six copy concentrations) are normalized by dividing by the number of assays in the relevant multiplex, Nᵢ. The number of partitions being positive for a particular signal (e.g., detectable wavelength, or “color”) can represent amplicons from multiple targets (each detected with the same color probe). In these embodiments, N, represents the number of multiple targets that are detected in an amplification set (and for example detected with probes having the same color label). In one illustrative embodiment, the average corrected concentration of fetal and maternal cfDNA may be found via interpolation within the hypermethylated and hypomethylated reference corrections, as shown in Equations 1 and 2.

$$[ {Fet}_{Corr} ] = ( \frac{[ Total ]}{[ Hyper ] - [ Hypo ]} ) * ( [ Fet ] - [ Hypo ] ) \qquad ( Eq . 1 )$$

$$[ {Mat}_{Corr} ] = ( \frac{[ Total ]}{[ Hyper ] - [ Hypo ]} ) * ( [ Mat ] - [ Hypo ] ) \qquad ( Eq . 2 )$$

wherein [Total] is total cfDNA copy concentration based on signal in partitions from the amplification set that targets total cfDNA comprising methylation insensitive regions from chromosomes unlikely to exhibit aneuploidy, [Hyper] is hypermethylated reference copy concentration based on signal in partitions from the amplification set that targets sites that are hypermethylated in fetal DNA and maternal DNA, [Hypo] is hypomethylated reference copy concentration based on signal in partitions from the amplification set that targets sites that are hypomethylated in fetal DNA and maternal DNA and [Fet] is fetal cfDNA copy concentration based on signal in partitions from the amplification set that targets sites that are hypermethylated in fetal DNA and hypomethylated in maternal DNA and [Mat] is maternal cfDNA copy concentration based on signal in partitions from the amplification set that target sites that are hypermethylated in maternal DNA and hypomethylated in fetal DNA.

Additionally, several relationships exist between independently measured metrics. Thus, adherence to the relationships expressed in Equations 3-5 can be used as measures of data quality.

$$[ Total ] = [ {Fet}_{Corr} ] + [ {Mat}_{Corr} ] \qquad ( Eq . 3 )$$

$$1 = FF + \frac{[ {Mat}_{Corr} ]}{[ Tot al ]} \qquad ( Eq . 4 )$$

$$2 * [ YChr ] = [ {Fet}_{Corr} ] , \qquad ( Eq . 5 )$$

wherein [YChr] is the concentration of Y-chromosome specific sequences based on signal in partitions from the amplification set that targets methylation-insensitive regions of the Y chromosome.

A male fetus may be determined by confirming that the Y chromosome copy concentration is non-zero and fulfills the relationship shown in Equation 5. Additionally, in the case of a Y chromosomal sex aneuploidy, the coefficient will be 1 instead of 2.

In the case of either a female or male fetus, the fetal fraction may be calculated, for example, by the four methods illustrated by Equations 6-9), which may be averaged to obtain a more precise value. Additionally, failure of the fetal fraction calculations to converge on an average value would indicate a problem with the data quality.

$$FF = \frac{[ {Fet}_{Corr} ]}{[ Total ]} \qquad ( Eq . 6 )$$

$$FF = \frac{[ {Fet}_{Corr} ]}{[ {Fet}_{Corr} ] + [ {Mat}_{Corr} ]} \qquad ( Eq . 7 )$$

$$FF = 1 - \frac{[ {Mat}_{Corr} ]}{[ Total ]} \qquad ( Eq . 8 )$$

$$FF = 1 - \frac{[ {Mat}_{Corr} ]}{[ {Fet}_{Corr} ] + [ {Mat}_{Corr} ]} \qquad ( Eq . 9 )$$

In the case of a male fetus, two additional fetal fraction calculations (Equations 10 and 11) become possible with the inclusion of data for the copy concentration of the Y chromosome.

$$F = \frac{[ YChr ]}{[ To tal ]} \qquad ( Eq . 10 )$$

$$FF = \frac{[ YChr ]}{[ {Fet}_{Corr} ] + [ {Mat}_{Corr} ]} \qquad ( Eq . 11 )$$

### Fetal Fraction Estimation Models

To increase the accuracy of the calculated fetal fraction, a model is developed to take in as input the calculated fetal fraction and outputs an adjusted fetal fraction based on the model. The model is developed with the objective of minimizing the average error of calculated fetal fractions from clinical samples with the next-generation sequencing (NGS) measurement of the fetal fractions of the same clinical samples. In one example, the model is designed to reduce the mean average percentage error, or the mean squared error.

For example, consider that x1, x2, x3, . . . , xN represent the calculated fetal fraction and z1, z2, z3, . . . , zN is corresponding NGS measurements, e.g., z1 is corresponding to x1, z2, is corresponding to x2, and so on. Further, consider the estimation model is represented as a function f( ) that takes in the calculated values x1, x2, . . . , xN, and for each calculated fraction computes an estimated fetal fraction y1, y2, y3, . . . , yN, e.g., y1=f(x1), y2=f(x2), . . . , yN=f(xN). An error for each estimated value can be defined as e1=(y1−z1)=(f(x1)−z1), e2=(y2−z2)=(f(x2)−z2), . . . eN=(yN−zN)=(f(xN)−zN). Several criteria may be considered in defining the estimation or mapping function f). For example, in minimizing the mean squared error (MSE), function f( ) is designed such that the MSE, e.g., (e1²+e2²+ . . . +eN²)/N, is minimized. In another example, f( ) may be designed such that the mean average absolute percentage error (MAPE), e.g., (|e1|/z1+|e2|/z2+ . . . +|eN|/zN)/N, where |.| is the absolute value operation, is minimized.

In one example, once the model is designed using the clinical or training data, the model may be used to estimate the fetal fraction based on the calculated fetal fraction. In another example, the model may be dynamic and can be updated using additional training data.

In one example, the function f(⋅) is a linear function. For example, f(x)=a x+b. In another example, a randomized noise may be included in the model, e.g., f(x)=a x+b+n, where n is a random variable having a predefined distribution.

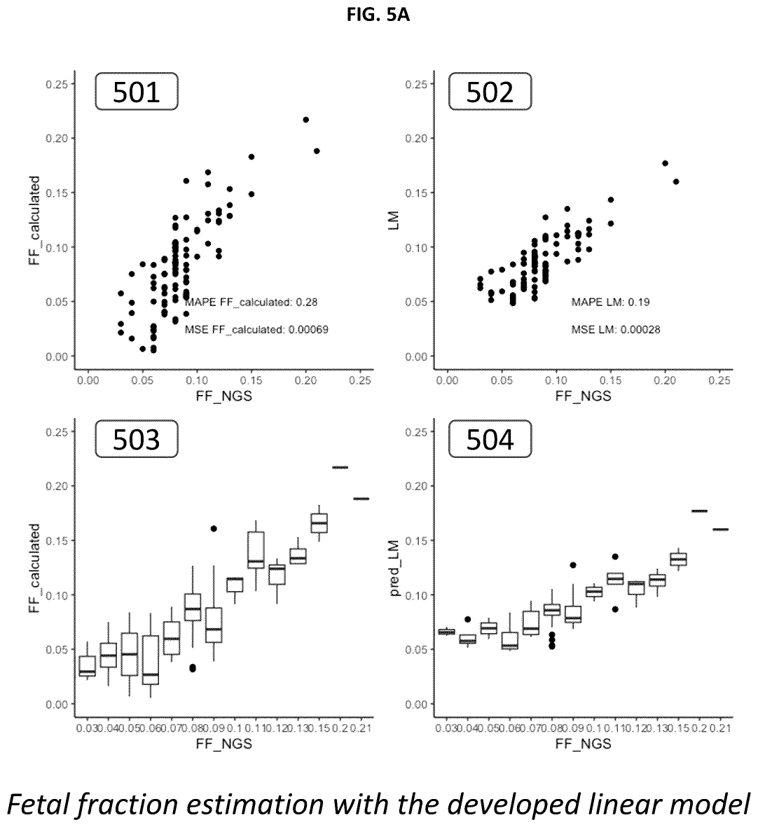

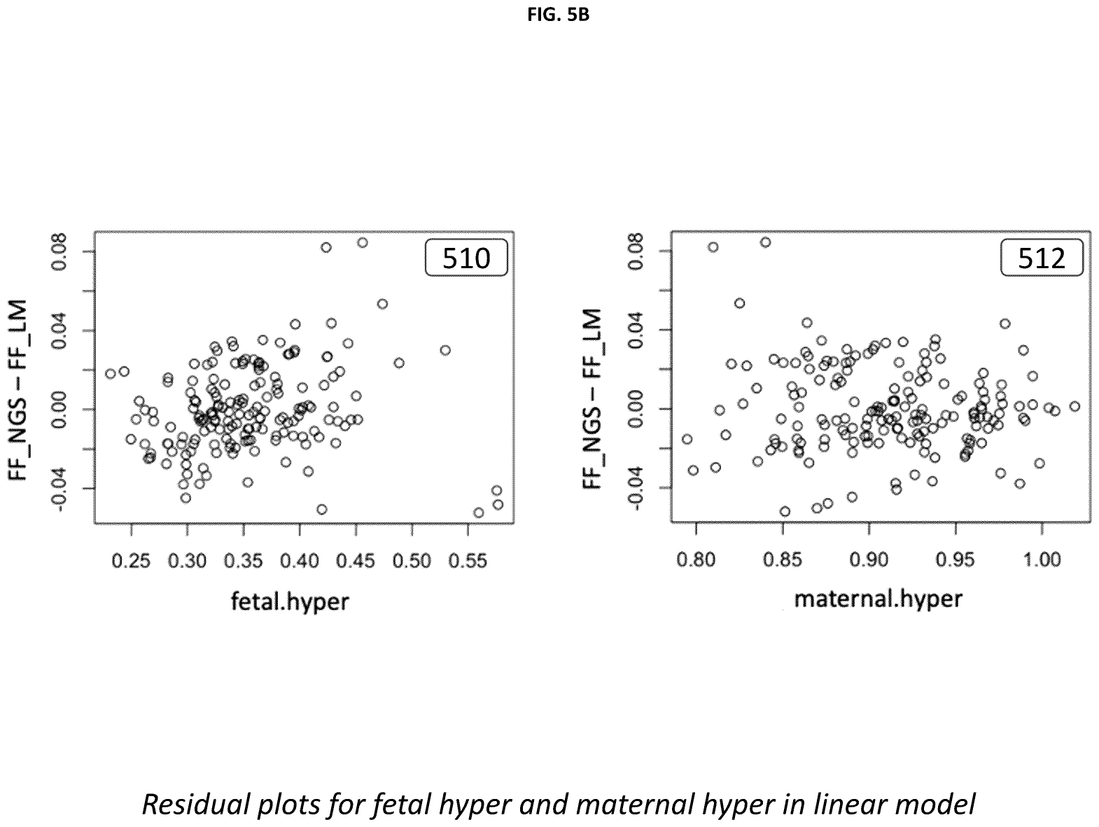

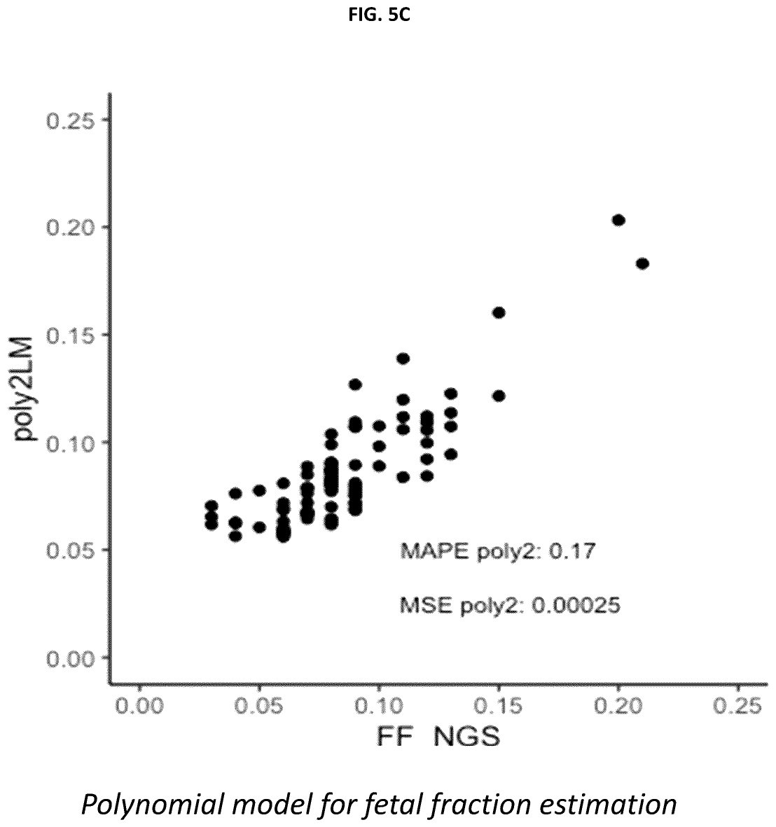

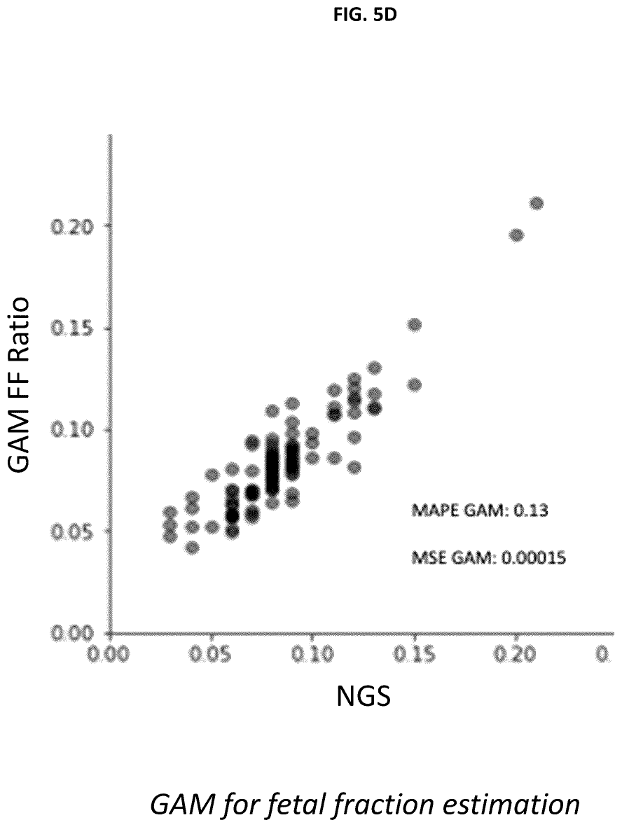

FIG. 5A illustrates the impact of applying a linear model on the calculated fetal fraction and its impact on MSE and MAPE. In 501, the calculated fetal fraction is depicted against the corresponding NGS fetal fraction. The calculated fetal fraction data has an MAPE of 28% and MSE of 6.9×10⁻⁴. In 503, the same data as in 501 is represented using boxes and whiskers. In 502, a linear model is applied to the calculated fetal fractions to obtain the depicted estimated fetal fractions. As the figure shows, the estimated values are more aligned with the corresponding NGS values, which is also validated by the reduced MSE and MAPE values. Using the linear model, the MAPE is reduced to 19% and the MSE to 2.8×10⁻⁴. In 504, the same data as in 502 is represented using boxes and whiskers. Comparing the boxes and whiskers in 504 and 503 shows the smaller variation in data and that the estimated fetal fraction is closer to the measured NGS values compared to the calculated fetal fractions.

In one example, the function f( ) may be a polynomial. For example, f(x)=a0+a1x+a2x²+a3x³+ . . . +aM x^{M}, where M determines the order of the polynomial. For example, M=2 for a second-order polynomial and f(x)=a0+a1x+a2x². Using optimization criteria, e.g., minimizing MSE or MAPE, and applying the model to the clinical data, coefficients a0, a1, and a2 can be computed.

FIG. 5B depicts the residual plots. In 510, the calculated fetal fraction is depicted against the fetal/hyper variable. Compared to the residual plot of the fetal/hyper, in 512, the calculated fetal fraction is depicted against the maternal/hyper variable. The plot does not suggest a linear relationship between the fetal/hyper and calculated fetal fraction.

FIG. 5C illustrates the impact of applying a linear model on the calculated fetal fraction and its impact on MSE and MAPE. The same calculated fetal fraction data as in 501 is used to develop a second-order polynomial model. The second-order polynomial model (poly2LM) is applied to the calculated fetal fractions to obtain the depicted estimated fetal fractions. As the FIG. 5C shows, the estimated values are more aligned along a line with the corresponding NGS values, which is also validated by the reduced MSE and MAPE values. Using the second-order polynomial model, the MAPE is reduced to 17% and the MSE to 2.5×10⁻⁴.

In one example, a generalized additive model (GAM) may be used to estimate the fetal fraction based on a calculated fetal fraction. GAM may be used to model non-linearity using an additive model. For example, a different relationship may exist between the calculated fetal fraction and the corresponding NGS values at different NGS values. A piece-wise model may be developed that defines a different estimator function. For example, the range of NGS can be divided into K regions, e.g., K=20, 30, or 40, and each region is estimated using a spline, e.g., a spline of order 2, 3, 4, or different orders. In addition, the GAM may incorporate other parameters as variables for the model, e.g., the gestational week, maternal weight, etc.

FIG. 5D illustrates the impact of applying GAM on the calculated fetal fraction and its impact on MSE and MAPE. The same calculated fetal fraction data as in 501 is used. The GAM is applied to the calculated fetal fractions to obtain the depicted estimated fetal fractions. As the FIG. 5D shows, the estimated values are more aligned along a line with the corresponding NGS values, which is also validated by the reduced MSE and MAPE values. Using GAM, the MAPE is reduced to 13% and the MSE to 1.5×10⁻⁴.

### Kits

In a further aspect, the disclosure provides a digital amplification kit for estimating the fraction of fetal DNA in a cfDNA sample obtained from a blood sample (such as a plasma or serum sample) from a pregnant subject, e.g., a human. The kit can include any of the components described herein with regard to the methods. In some embodiments, the kit comprises an amplification reaction mixture comprising amplification reagents, and a plurality of amplification sets comprising primer and probe sets, wherein each amplification set comprises a distinct label distinguishable from the label for each of the other sets, and each set comprises primers and probes for multiplex amplification, and wherein the plurality of amplification sets comprises (i) an amplification set that targets sites that are hypermethylated in fetal DNA and hypomethylated in maternal DNA; and/or (ii) an amplification set that targets sites that are hypermethylated in maternal DNA and hypomethylated in fetal DNA. In some embodiments, the kit further comprises an amplification set that targets total cfDNA comprising methylation insensitive regions from chromosomes unlikely to exhibit aneuploidy. In some embodiments, the kit comprises an amplification set that targets sites that are hypermethylated in fetal DNA and maternal DNA; and/or an amplification set that targets sites that are hypomethylated in fetal DNA and maternal DNA. In some embodiments, the kit further comprises an amplification set that targets methylation-insensitive regions of the Y chromosome. In some embodiments, one or more sites are selected from Table 6 as described elsewhere herein. In some embodiments, a kit as described in this paragraph comprises a completely methylated synthetic sequence of DNA and/or a completely unmethylated version of the same synthetic sequence. In some embodiments, the probes employed for detection are molecular beacon probes, e.g., fluorescently labeled molecular beacon probes.

In some embodiments, each of the amplification sets of (i) and (ii) comprises primers and probes to target at least two sites, at least three sites, or more, for example, 1-20 sites, e.g., or between 6-10 sites.

In some embodiments, each label for an amplification set is a fluorescent label. In some embodiments, one or more primers or a probe comprises a region that does not hybridize to the target amplification site but is complementary to an oligonucleotide that provides a detectable signal.

In some embodiments, a kit comprising one or more amplification sets as described in the preceding paragraphs further comprises at least one methylation-sensitive restriction enzyme that cleaves hypomethylated DNA. In some embodiments, the kit comprises one or more of the restriction enzymes, HhaI, HpaII, AciI, HpyCH4IV, or BsaHI. In some embodiments, a methylation-sensitive restriction enzyme (MSRE) cocktail comprises at least one, two, at least three, or at least four methylation-sensitive restriction enzymes; selected from HhaI, HpaII, AciI, HpyCH4IV, and BsaHI. In some embodiments, the kit comprises a methylation-sensitive restriction enzyme (MSRE) cocktail comprising at least two, three, or more of the restriction enzymes HhaI, HpaII, AciI, HpyCH4IV, and BsaHI. In some embodiments, the MSRE cocktail comprises at least HhaI and HpyCH4IV.

In some embodiments, the reaction mixture is lyophilized.

In some embodiments, a kit comprises a standard ddPCR kit, sets of primers and probes for a fetal fraction assay as described herein, and at least one MSRE. In some embodiments, such a kit further comprises at least one PCR-enhancing agent such as TMAC and/or salts.

In some embodiments, a kit comprises a standard ddPCR kit, sets of primers and probes for a fetal fraction assay as described herein, at least one MSRE, and stabilizing agents (e.g., trehalose, potassium glutamate, ammonium sulfate) (lyophilized together in one mix).

In some embodiments, a kit comprises a standard ddPCR kit, sets of primers and probes for a fetal fraction assay as described herein; stabilizing reagents (lyophilized together) and at least one MSRE, which may or may not be lyophilized.

### EXAMPLES

The following examples further illustrate aspects and embodiments of the methods of the present disclosure.

### Example 1: A Methylation-Based Digital PCR Assay for Fetal Cell-Free DNA Quantification in NIPT Samples

CfDNA is first isolated from maternal blood plasma, e.g., using a commercially available kit such as a magnetic bead kit designed for preferential capture and elution of cfDNA (e.g., a magnetic bead-based extraction kit (Apostle)). The fragment length distribution of the cfDNA eluate may be confirmed, e.g., via a commercially available method, such as a High-Sensitivity DNA Bioanalyzer kit (Agilent).

CfDNA isolated from the maternal sample is digested with a methylation-sensitive restriction enzyme (MSRE) cocktail containing enzymes such as, but not limited to, HhaI, HpaII, AciI, or HpyCH4IV. These enzymes cleave DNA at sites where several bases are present in a specific sequence, and only if the sites are not methylated (Table 1). For differentially methylated sites (DMSs) that are hypermethylated in fetal cfDNA and hypomethylated in maternal cfDNA, only fetal cfDNA DMSs remain to be quantified. Conversely, for DMSs that are hypermethylated in maternal cfDNA and hypomethylated in fetal cfDNA, only maternal cfDNA DMSs remain for quantitation. While in some embodiments, the digestion of cfDNA can be conducted in the bulk, in this illustrative embodiment, digestion is performed after partitioning of the sample and PCR reagents, but before PCR amplification.

The cfDNA (e.g., eluate from the sample) is added to a dPCR reaction mix containing a dPCR supermix (including all reagents necessary for both PCR and partitioning), MSRE cocktail, and in this example, a 6-channel assay set (Table 1). This in-partition digestion technique allows for a more streamlined procedure and single thermocycling run (Table 2).

A methylation-sensitive digestion strategy described herein is further illustrated below, using a 4-channel droplet digital PCR (ddPCR) instrument (QX ONE).

Twenty-two maternal plasma samples (CerbaXpert, France), with fetal fractions ranging between 10 and 25% as measured by VeriSeq NIPT, were selected along with two non-pregnant plasma samples (Stanford Blood Bank). cfDNA was extracted from the twenty-four samples using the Apostle MiniMax High Efficiency cfDNA Isolation Kit, performed in an automated fashion on the KingFisher Flex (ThermoFisher). Prior to use with ddPCR, the cfDNA was characterized on the Bioanalyzer platform (Agilent) and found to contain 62%±6% mononucleosomal cfDNA, while the remaining nucleic acid content was comprised of high molecular weight genomic DNA.

For each sample, 5.5 μL of the extraction eluate was used in six 22-μL, ddPCR reactions (Table 4). All reactions contained a fetal DMS triplex (FAM channel) targeting sites that are hypermethylated in fetal cfDNA and hypomethylated in maternal cfDNA, a maternal DMS triplex (HEX channel) targeting sites that are hypermethylated in maternal cfDNA and hypomethylated in fetal cfDNA, and a Y chromosome target (SRY, Cy5.5 channel). In the remaining Cy5 channel, three of the reactions contained an X chromosome target (SPIN4) and three of the reactions contained a chromosome 10 target (RPP30). Additionally, four reactions contained an MSRE cocktail (HhaI and HpyCH4IV, 10U each per ddPCR reaction) while two reactions did not.

The fetal and maternal triplexes were used to calculate fetal fraction as shown in Equation 6-11, albeit using uncorrected values. The copy concentrations of SRY, SPIN4, and RPP30 were used to calculate orthogonal ddPCR fetal fraction estimates that do not rely on the methylation-sensitive digestion scheme.

Results

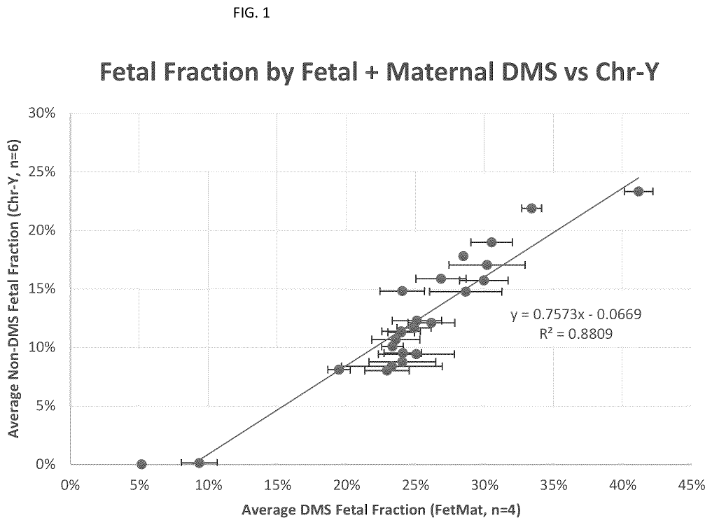

FIG. 1 shows correlation between the methylation-sensitive fetal fraction estimations and the SRY-based fetal fraction estimations for the 24 samples. When both the SRY/SPIN4 and SRY/RPP30 calculations are included, the R² of the positive correlation is 0.88. Additionally, the R² of the correlation between the methylation-sensitive fetal fraction estimation and the VeriSeq noninvasive prenatal testing (NIPT) determination is 0.84 (Table 5).

Use with Other Fetal Diagnostic Assays

With sufficient multiplexing, this fetal fraction quantification method can be used simultaneously with other fetal cfDNA tests, including trisomy and microdeletion detections. In some embodiments the assay can be conducted alongside these tests in separate wells using the same sample, since the present methods only employ a fraction of cfDNA obtained from a maternal sample. In some embodiments, if a multiple-well test is desired, conducting MSRE digestion in-droplet for fetal fraction determination simplifies the workflow. In this instance, cfDNA eluate is added to each test well, and only cfDNA used in fetal fraction determination would be digested due to the presence of MSREs in the fetal fraction test reaction mix. In some embodiments the assay can be conducted alongside diagnostic tests for single gene disorders, such as but not limited to alpha- or beta-thalassemia, cystic fibrosis or hemophilia A.

### Example 2: A Highly Multiplexed Methylation-Based ddPCR Assay and Machine Learning Methods for Fetal Cell-Free DNA Fraction Determination in NIPT Samples

In this example, a highly multiplexed methylation-based ddPCR assay, along with machine learning-based methods were utilized for the analysis of fetal cfDNA fraction in NIPT samples.

Fetal fraction (FF) refers to the portion of fetal cfDNA in a pregnant mother's blood. The estimation of the fetal fraction can be used, for example, to assess fetal aneuploidy in a non-invasive way. Currently, next-generation sequencing (NGS) performs as the gold-standard method for fetal fraction estimation. Approaches include profiling single-nucleotide polymorphisms to analyze the different genotypes between the fetus and mother, measuring the proportion of chromosome Y cfDNA reads for male fetus, and examining the differences in methylation profiles. However, sequencing-based approaches are not cost-efficient. Digital droplet PCR (ddPCR) involves partitioning individual PCR reactions into droplets, allowing for high levels of sensitivity and accuracy, as well as reduced cost relative to NGS.

In Example 1, a methylation-based ddPCR assay was designed based on the different methylation patterns between maternal and fetal cfDNA in NIPT samples to calculate the fetal fraction. This example describes a highly multiplexed methylation-based ddPCR assay to simultaneously quantify fetal and maternal cfDNA in the same ddPCR reaction with improved accuracy for quantification of fetal fraction in NIPT samples.

ddPCR Assay Design and Machine Learning Methods

The ddPCR assay used 6 fluorescence channels (QX600 instrument, Bio-Rad Laboratories), with between 6-10 targets per channel, targeting:
(i) sites hypermethylated in fetal cfDNA and hypomethylated in maternal cfDNA to target fetal cfDNA after methylation sensitive restriction enzyme digestion (“Fetal”);
(ii) sites hypermethylated in maternal cfDNA and hypomethylated in fetal cfDNA to target maternal cfDNA after methylation sensitive restriction enzyme digestion (“Maternal”);
(iii) sites hypomethylated in fetal and maternal cfDNA (“Hypo”) to target methylation sensitive enzyme digestion efficiency together with “Hyper” as explained in (iv);
(iv) sites hypermethylated in fetal and maternal cfDNA (“Hyper”) to target methylation sensitive enzyme digestion with “Hypo”;
(v) sites on chromosome Y (“Chr Y”) not containing CpG sites (i.e., methylation insensitive regions) for male fetus; and
(vi) control sites (“Control”) not containing CpG sites for the control of the assay performance.

The estimation of the fetal cfDNA fraction was performed with fetal/hyper, maternal/hyper, hypo/hyper, and chrY/control. The fetal/hyper, maternal/hyper, hypo/hyper, and chrY/control were calculated based on the lambda ratio of the “Fetal” divided by “Hyper”, lambda ratio of the “Maternal” divided by “Hyper”, lambda ratio of the “Hypo” divided by “Hyper”, and lambda ratio of the “chrY” divided by “control”.

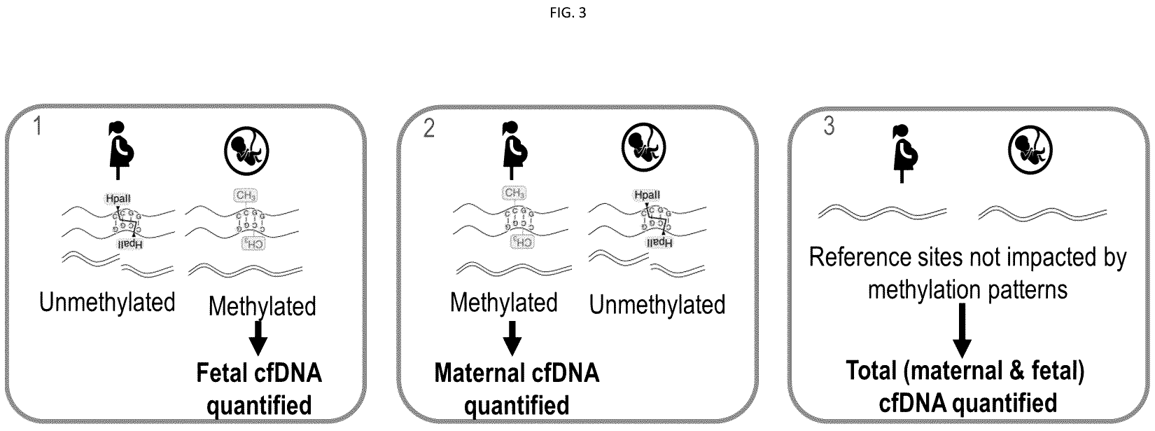

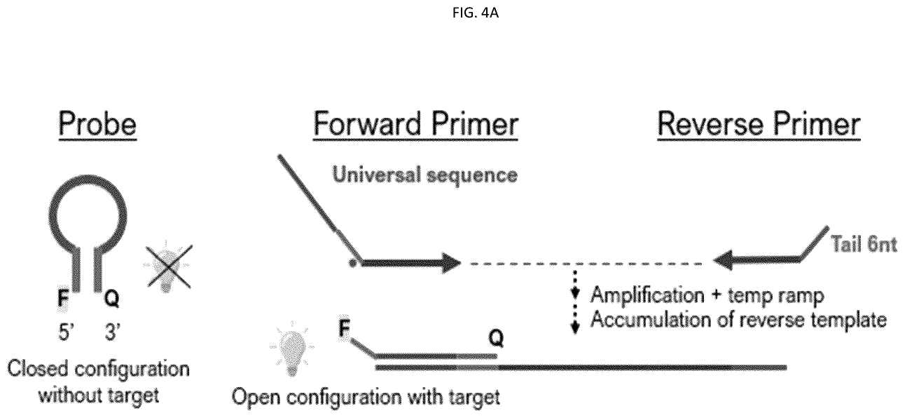

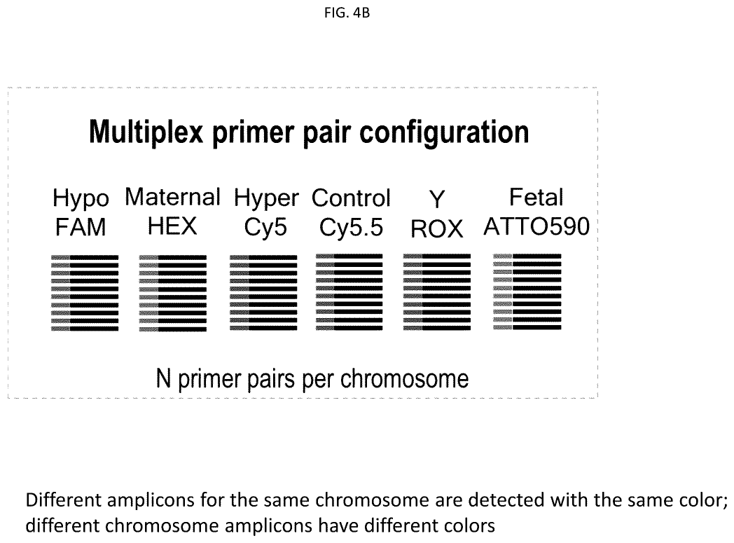

An overview of the design of the assay is provided in FIGS. 3 and 4A-4B. Table 6 provides exemplary target sites that can be used for fetal fraction quantification in this ddPCR assay.

To further improve the assessment of the fetal fraction, machine learning linear regression, polynomial, and generalized additive models trained on clinical NIPT samples were developed. Due to the limitation of the clinical NIPT samples, over 70 samples were utilized to train the three models. The fetal/hyper, maternal/hyper, hypo/hyper and chrY/control calculated from the highly multiplex fetal fraction assay as mentioned in Table 6 functioned as input variables of the three models, while other metadata from the clinical NIPT samples, e.g., gestational weeks, were also tested to improve the performance of the models.

Results

As shown, in FIGS. 5A-5D and Table 7, the mean average percentage error (MAPE) and mean squared error (MSE) of the ddPCR-based fetal fraction estimates relative to NGS-based calculations were dramatically decreased when using the linear regression, polynomial, and generalized additive models, as compared to prior analysis methods.

For example, fetal fraction estimation using a linear regression model (“LM”) that was trained by clinical samples had smaller error compared to fetal fraction calculation using Equations 1-11 (“FF_calculated”), including smaller MAPE and MSE (see FIG. 5A and Table 7). The relationship between the fetal/hyper and maternal/hyper variables with the fetal fraction using NGS and the linear model was illustrated in FIG. 5B. To further improve the model, the residual plots were utilized to decipher the relationships between the fetal/hyper, maternal/hyper and fetal fraction using NGS. The plots in FIG. 5B show a non-linear relationship between fetal fraction and the fetal/hyper compared with the maternal/hyper variables. Using a second-order term of fetal/hyper in the polynomial model may improve the performance measures, e.g., MAPE and MSE. For example, FIG. 5C illustrates the estimated fetal fraction using a second-order polynomial model (“poly2LM”). The second-order polynomial model had smaller error compared to prior methods, including smaller MAPE and MSE (see FIGS. 5A, 5C and Table 7).

The generalized additive model (GAM) is a generalized linear model that can model non-linear data with interpretability. The GAM had smaller errors compared to prior methods, including smaller MAPE and MSE (see FIG. 5D and Table 7).

The ddPCR assay and fetal fraction calculation methods described in this example show great promise in improving the accuracy and reliability of ddPCR in fetal fraction estimation, which contributes to ddPCR aneuploidy NIPT tests, making it a useful tool for prenatal screening and diagnosis.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

TABLE 1
Example of dPCR Master Mix for In-Partition Digestion

| Component | Volume/μL |
|---|---|
| dPCR Multiplex Supermix, 4X | 5.0 |
| 4-enzyme MSRE Cocktail (5 U/μL) | 2.0 |
| Fetal DMS Multiplex Assay, 40X (FAM) | 0.5 |
| Maternal DMS Multiplex Assay, 40X (HEX) | 0.5 |
| Total cfDNA Multiplex Assay, 40X (Cy5) | 0.5 |
| Y Chromosome Multiplex Assay, 40X (Cy5.5) | 0.5 |
| Hypermethylated Reference Assay, 40X (Rox) | 0.5 |
| Hypomethylated Reference Assay, 40X (Atto) | 0.5 |
| cfDNA Eluate | 9.0 |
| Total | 20.0 |

TABLE 2
ddPCR MSRE Digestion and Thermocycling Protocol

| Step | Temperature/C. | Time | | Cycles |
|---|---|---|---|---|
| MSRE Digestion | 37 | 45 | minutes | 1 |
| Taq HotStart/ | 95 | 10 | minutes | 1 |
| MSRE | | | | |
| Inactivation | | | | |
| Denaturation | 95 | 30 | seconds | 40 |
| Anneal/ | 59 | 2 | minutes | |
| Extension | | | | |
| Taq Inactivation | 95 | 5 | minutes | 1 |
| Stabilization | 4 | 5 | minutes | 1 |

TABLE 3
Example of dPCR Assay Multiplexes and Copy Concentration Readouts

| | | Copy | |
|---|---|---|---|
| | | Concentration | |
| Assay | Channel | Value (cp/μL) | Description |
| Fetal DMS | FAM | [Fet] * N_{Fet} | Fetal cfDNA copy concentration |
| | | | multiplied by number of assays in |
| | | | multiplex |
| Maternal DMS | HEX | [Mat] * N_{Mat} | Maternal cfDNA copy concentration |
| | | | multiplied by number of assays in |
| | | | multiplex |
| Total cfDNA | Cy5 | [Total] * N_{Total} | Total cfDNA copy concentration |
| | | | multiplied by number of assays in |
| | | | multiplex |
| Y Chromosome | Cy5.5 | [YChr] * N_{YChr} | Y chromosome copy concentration |
| | | | multiplied by number of assays in |
| | | | multiplex |
| Hypermethylated | Rox | [Hyper] * N_{Hyper} | Hypermethylated reference copy |
| Reference | | | concentration multiplied by number of |
| | | | assays in multiplex |
| Hypomethylated | Atto | [Hypo] * N_{Hypo} | Hypomethylated reference copy |
| Reference | | | concentration multiplied by number of |
| | | | assays in multiplex |

TABLE 4
MSRE and Reference Target Scheme

| Reaction | MSRE | SPIN4 | RPP30 |
|---|---|---|---|
| 1 | Yes | Yes | No |
| 2 | Yes | Yes | No |
| 3 | Yes | No | Yes |
| 4 | Yes | No | Yes |
| 5 | No | Yes | No |
| 6 | No | No | Yes |

TABLE 5
Correlation of Methylation-Sensitive Digestion
Fetal Fraction with Orthogonal Measures.

| Orthogonal Measure | Coefficient R2 |
|---|---|
| SRY with SPIN4 (n = 3) | 0.74 |
| SRY with RPP30 (n = 3) | 0.87 |
| SRY with SPIN4 & RPP30 (n = 3) | 0.88 |
| VeriSeq NIPT | 0.84 |

TABLE 6
Exemplary Fetal Fraction methylation-based ddPCR assay
target regions as set forth in genome build 38.

| Target | Chromosome | Start | End |
|---|---|---|---|
| fetal | chr1 | 10965026 | 10965118 |
| fetal | chr1 | 36457885 | 36458000 |
| fetal | chr11 | 77152247 | 77152348 |
| fetal | chr14 | 100081619 | 100081733 |
| fetal | chr14 | 105390470 | 105390583 |
| fetal | chr15 | 81781663 | 81781753 |
| fetal | chr17 | 75997501 | 75997633 |
| fetal | chr2 | 196211575 | 196211683 |
| hyper | chr19 | 12930395 | 12930501 |
| hyper | chr2 | 98903445 | 98903560 |
| hyper | chr20 | 50670188 | 50670287 |
| hyper | chr4 | 186620052 | 186620153 |
| hyper | chr4 | 41181277 | 41181358 |
| hyper | chr5 | 133097153 | 133097246 |
| hyper | chr6 | 3363036 | 3363147 |
| hypo | chr10 | 73744741 | 73744849 |
| hypo | chr12 | 104050025 | 104050120 |
| hypo | chr17 | 48108167 | 48108297 |
| hypo | chr17 | 68292301 | 68292434 |
| hypo | chr2 | 27369852 | 27369937 |
| hypo | chr2 | 28870489 | 28870604 |
| maternal | chr1 | 110390715 | 110390806 |
| maternal | chr19 | 8525569 | 8525674 |
| maternal | chr2 | 236576106 | 236576215 |
| maternal | chr2 | 239532002 | 239532126 |
| maternal | chr2 | 72115414 | 72115526 |
| maternal | chr4 | 90104102 | 90104228 |
| maternal | chr5 | 142280348 | 142280462 |
| Y | chrY | 13817626 | 13817726 |
| Y | chrY | 13861919 | 13862016 |
| Y | chrY | 14225706 | 14225797 |
| Y | chrY | 14405980 | 14406112 |
| Y | chrY | 15558950 | 15559068 |
| Y | chrY | 16701419 | 16701541 |
| Y | chrY | 17109321 | 17109424 |
| Y | chrY | 19228514 | 19228627 |
| Y | chrY | 19731092 | 19731204 |
| Y | chrY | 21046467 | 21046568 |
| control | chr3 | 104042356 | 104042470 |
| control | chr3 | 104263905 | 104264029 |
| control | chr3 | 133296976 | 133297077 |
| control | chr3 | 169019731 | 169019836 |
| control | chr3 | 17807278 | 17807410 |
| control | chr3 | 22594415 | 22594519 |
| control | chr3 | 25620061 | 25620168 |
| control | chr3 | 35697692 | 35697774 |
| control | chr3 | 82529219 | 82529320 |

TABLE 7
Performance of fetal fraction estimation in different algorithms.

| | MAPE | MSE |
|---|---|---|
| Baseline method | 0.28 | 0.00069 |
| Linear model | 0.19 | 0.00028 |
| Polynomial model | 0.17 | 0.00025 |
| GAM | 0.13 | 0.00015 |

What is claimed is:

1. A method of estimating the fraction of fetal DNA in a cfDNA sample obtained from a blood sample from a pregnant human subject, the method comprising a digital amplification reaction method comprising:
(a) partitioning into partitions an amplification reaction mixture comprising cfDNA from the cfDNA sample, amplification reagents, and a plurality of amplification sets comprising primer and probe sets, wherein each amplification set comprises primers and probes for multiplex amplification and each amplification set generates amplification products, when target is present, comprising a distinct label distinguishable from the label for each of the other amplification sets; and wherein the plurality comprises:
(i) an amplification set that targets sites that are hypermethylated in fetal DNA and hypomethylated in maternal DNA; and
(ii) an amplification set that targets sites that are hypermethylated in maternal DNA and hypomethylated in fetal DNA; and optionally one or more of (iii), (iv), and (v):
(iii) an amplification set that targets total cfDNA comprising methylation insensitive regions from chromosomes unlikely to exhibit aneuploidy;
(iv) an amplification set that targets sites that are hypermethylated in fetal DNA and maternal DNA;
(v) an amplification set that targets sites that are hypomethylated in fetal DNA and maternal DNA;
(b) incubating the cfDNA with a methylation-sensitive restriction enzyme (MSRE) cocktail comprising at least one methylation-sensitive restriction enzyme that cleaves unmethylated DNA;
(c) amplifying target nucleic acid sequences in the partitions, if present, to obtain amplification products;
(d) detecting in the partitions a signal from each distinct label from the amplification products; and
(e) quantifying the signal for each distinct label.

2. The method of claim 1, wherein the plurality comprises (iii) the amplification set that targets total cfDNA comprising methylation insensitive regions from chromosomes unlikely to exhibit aneuploidy.

3. The method of claim 1, wherein each of the amplification sets of (i) and (ii) comprises primers and probes to target at least three sites.

4. The method of claim 1, wherein each of the amplification sets of (i)-(v) comprises primers and probes to target at least three sites or between 6-10 sites.

5. The method of claim 1, further comprising an amplification set that targets methylation-insensitve regions of the Y chromosome.

6. The method of claim 1, further comprising an amplification set that targets sites that are hypomethylated in both fetal and maternal cfDNA; and/or an amplification set that targets sites that are hypermethylated in both fetal and maternal cfDNA.

7. The method of claim 1, wherein the amplification reaction mixture further comprises a control target completely methylated synthetic DNA sequence and/or a completely unmethylated version of the same synthetic DNA sequence.

8. The method of claim 1, wherein the digital amplification reaction method is a digital PCR method.

9. The method of claim 8, wherein the digital PCR method is a droplet digital PCR method.

10. The method of claim 1, wherein the amplification reaction mixture comprises the MSRE cocktail, and the incubating occurs after the partitioning and before the amplifying.

11. The method of claim 1, wherein step (b) is performed before the partitioning and cfDNA subjected to digestion is added to the amplification reaction mixture.

12. The method of claim 1, wherein the MSRE cocktail comprises at least two, at least three, or at least four methylation-sensitive restriction enzymes; and/or wherein the MSRE cocktail comprises a restriction enzyme sleeted from HhaI, HpaII, AciI, HpyCH4IV, and BsaHI.

13. The method of claim 1, wherein the cfDNA sample is obtained from plasma or serum.

14. The method of claim 1, further comprising determining the normalized copy concentration for each of the targets, based on the number of targets in an amplification set (Nᵢ).

15. The method of claim 1, further comprising determining a corrected concentration of fetal cfDNA (Fet_{Corr}) in the cfDNA sample and/or a corrected concentration of maternal cfDNA (Mat_{Corr}) in the cfDNA sample, wherein determining the corrected concentration of fetal cfDNA comprises a calculation:
$$[ {Fet}_{Corr} ] = ( \frac{[ Total ]}{[ Hyper ] - [ Hypo ]} ) * ( [ Fet ] - [ Hypo ] ) ; \qquad ( Eq . 1 )$$
wherein [Total] is total cfDNA copy concentration based on signal in partitions from the amplification set that targets total cfDNA comprising methylation insensitive regions from chromosomes unlikely to exhibit aneuploidy, [Hyper] is hypermethylated reference copy concentration based on signal in partitions from the amplification set that targets sites that are hypermethylated in fetal DNA and maternal DNA, [Hypo] is hypomethylated reference copy concentration based on signal in partitions from the amplification set that targets sites that are hypomethylated in fetal DNA and maternal DNA and [Fet] is fetal cfDNA copy concentration based on signal in partitions from the amplification set that targets sites that are hypermethylated in fetal DNA and hypomethylated in maternal DNA;
and determining the corrected concentration of maternal cfDNA comprises a calculation:
$$[ {Mat}_{Corr} ] = ( \frac{[ Total ]}{[ Hyper ] - [ Hypo ]} ) * ( [ Mat ] - [ Hypo ] ) , \qquad ( Eq . 2 )$$
wherein [Mat] is maternal cfDNA copy concentration based on signal in partitions from the amplification set that targets sites that are hypermethylated in maternal DNA and hypomethylated in fetal DNA.

16. The method of claim 1, further comprising determining the fetal fraction (FF) in the cfDNA sample, wherein determining the fetal fraction comprises at least one of the following calculations (a)-(d):
$$FF = \frac{[ {Fet}_{Corr} ]}{[ Total ]} \qquad ( a )$$
$$FF = \frac{[ {Fet}_{Corr} ]}{[ {Fet}_{Corr} ] + [ {Mat}_{Corr} ]} , \qquad ( b )$$
$$FF = 1 - \frac{[ {Mat}_{Corr} ]}{[ Total ]} , or \qquad ( c )$$
$$FF = 1 - \frac{[ {Mat}_{Corr} ]}{[ {Fet}_{Corr} ] + [ {Mat}_{Corr} ]} . \qquad ( d )$$

17. The method of claim 16, further comprising:
computing an estimated fetal fraction at least partially based on the fetal fraction in the cfDNA sample and a model.

18. The method of claim 17, wherein the model is a generalized additive model (GAM), a linear model, or a second-order polynomial model at least partially based on a set of clinical fetal fraction data and a corresponding set of fetal fraction measurements using next-generation sequencing (NGS).

19. The method of claim 5, further comprising determining the fetal fraction of a male fetus in the cfDNA sample, wherein determining the male fetus fetal fraction comprises:
$$FF = 1 - \frac{[ {Mat}_{Corr} ]}{[ {Fet}_{Corr} ] + [ {Mat}_{Corr} ]} or \qquad ( Eq . 10 )$$
$$FF = \frac{[ YChr ]}{[ To tal ]} or$$
$$FF = \frac{[ YChr ]}{[ {Fet}_{Corr} ] + [ {Mat}_{Corr} ]} ,$$
wherein [YChr] is the concentration of Y-chromosome specific sequences based on signal in partitions from the amplification set that targets methylation-insensitive regions of the Y chromosome.

20. A digital amplification kit for estimating the fraction of fetal DNA in a cfDNA sample obtained from a plasma or serum sample from a pregnant human subject, the kit comprising:
(a) an amplification reaction mixture comprising amplification reagents, and a plurality of amplification sets comprising primer and probe sets, wherein each amplification set comprises a distinct label distinguishable from the label for each of the other sets, and each set comprises primers and probes for multiplex amplification, and wherein the plurality of comprises:
(i) an amplification set that targets sites that are hypermethylated in fetal DNA and hypomethylated in maternal DNA; and
(ii) an amplification set that targets sites that are hypermethylated in maternal DNA and hypomethylated in fetal DNA; and optionally one or more of (iii), (iv), and (v);
(iii) an amplification set that targets total cfDNA comprising methylation insensitive regions from chromosomes unlikely to exhibit aneuploidy;
(iv) an amplification set that targets sites that are hypermethylated in fetal DNA and maternal DNA;
(v) an amplification set that targets sites that are hypomethylated in fetal DNA and maternal DNA.
